(19) United States
(12) Patent Application Publication
(10) Pub. No.: US 2021/0299182 A1
(43) Pub. Date: Sep. 30, 2021

(54) METHODS AND SYSTEMS FOR PRODUCING AND ADMINISTERING ANTIVIRAL PLATELET THERAPY

(71) Applicant: Bridging Biosciences, LLC, Frisco, TX (US)

(72) Inventors: Tapley Holland, Frisco, TX (US); Huston Davis Adkisson, St. Louis, MO (US)

(21) Appl. No.: 17/217,820

(22) Filed: Mar. 30, 2021

Related U.S. Application Data
(60) Provisional application No. 63/002,093, filed on Mar. 30, 2020.

(51) Int. Cl. A61K 35/19; A61K 35/16; A61K 9/00

(52) U.S. Cl. CPC A61K 35/19; A61K 2035/124; A61K 9/0019; A61K 35/16

(57) ABSTRACT

This invention relates in general to the field of cell-therapy treatments and more particularly, but not by way of limitation, to systems and methods for administering personalized cell-therapy treatments intravenously. In various embodiments, the system may calculate an aspiration volume needed for centrifugation to achieve a concentrated target threshold dose of 2.5×10⁶ platelets/μL for a particular cell therapy using various factors such as, for example, information about a patient and the efficiency of the concentration process.

### CROSS-REFERENCE TO RELATED APPLICATIONS

This patent application is related to U.S. patent application Ser. No. 16/679,107, filed Nov. 8, 2019, which is hereby incorporated by reference for all purposes.

### BACKGROUND

### Technical Field

The present invention relates to treatment methods and protocols for the administration of supra-physiologic Platelet Rich Plasma (PRP) in the treatment of disease conditions resulting from viral pathogen invasion.

### Background

Conventional wisdom teaches that blood platelets function primarily to maintain hemostasis and thrombosis, whereas white blood cells are responsible for immune defense. While platelets are recognized to contribute to the healing cascade following tissue injury, the clinical benefits of platelet concentrate (PRP) have yielded variable results largely related to the lack of standardization of both its preparation and route of administration. Recent scientific and clinical evidence has conclusively demonstrated the integral role with which platelets may contribute to the immune response in various types of infection, including bacterial and viral pathogens. Bone marrow is historically believed to be the primary site for platelet production; however, new experimental evidence suggests a novel mechanism that lays the foundation for new discoveries in clinical immunology.

As the organ with the greatest surface area, the lung serves as a critical immune interface protecting against invading pathogens. The airway is endowed with a broad armamentarium of cellular and humoral host defense mechanisms, most of which belong to the innate arm of the immune system. Once thought to orchestrate the complex interplay between resident and infiltrating immune cells acting in concert with secreted innate immune proteins, airway epithelial cells may now begin to yield this position to lung-derived platelets, which have only recently emerged as primary effector cells sensing and responding to invading pathogens.

Platelets, small (2-5 micron) anucleated cell fragments derived from megakaryocytes, exhibit a normal lifespan of 7-10 days and circulate in high numbers, representing the most abundant cellular component of blood. Platelets are produced during hematopoiesis in a sub-process called thrombopoiesis, or production of thrombocytes. Thrombopoiesis occurs from common myeloid progenitor cells in the bone marrow, which differentiate into promegakaryocytes followed by mature megakaryocytes. Mature megakaryocytes produce platelets by cytoplasmic fragmentation occurring through a highly regulated, dynamic process called proplatelet formation and consisting of long pseudopodial elongations within capillaries that fragment under local sheer forces.

While platelet biogenesis initially was proposed to occur predominantly within the bone marrow niche, through an analysis of platelets entering and exiting the lungs, prior research proposed as early as 1937 that an alternative organ—the lung—represents a novel location supporting significant platelet production, estimated to represent ⅓ of total circulating platelets.

This fundamental discovery remained hidden within the scientific literature for greater than 80 years until only recently when it was validated, providing unequivocal evidence as to the previously unrecognized role of the pulmonary system in hematopoietic function during platelet biogenesis. Through an analysis of live cell imaging and orthotopic lung transplantation, it has been reported that in fact 50% of the total population of circulating platelets (10 million per hour) originates from megakaryocytes that actively migrate to the pulmonary interstitium from bone marrow to spawn mature platelets.

The significance of lung involvement in the regulation of platelet biogenesis and how this mechanism may ultimately impact clinical medicine remains poorly understood. Applicants were some of the first to hypothesize that the pulmonary microenvironment may offer a primary axis for immune surveillance and pathogen destruction specifically involving platelets, much like the gut-brain axis wherein sentinel lymphocytes play an essential role in the maintenance of host defenses. Importantly, when lung function is compromised by disease, either a reduction in normal fluid flow within alveolar capillaries or biologic dysfunction in the lung microenvironment may produce failed megakaryocyte maturation that ultimately yields a reduction in platelet biogenesis. Furthermore, it is likely that a combination of biologic and mechanical dysfunction within lungs exhibiting compromised pulmonary function (during aging and disease) disrupts pro-platelet maturation and release leading to immune insufficiency.

Within the context of the lung, immune insufficiency consequently predisposes certain groups of individuals incapable of evoking natural immune effector responses to viral pathogen invasion, including identification, packaging, and subsequent neutrophil signaling, for efficient clearance of the pathogen. These specific patients may include individuals with COPD, idiopathic pulmonary fibrosis, asthma, vaping lung injury, high dose chemotherapy, lung radiation, allograft transplantation, idiopathic thrombocytopenia, and subjects with chronic history of smoking. As will be discussed later, there is evidence this scenario may in part contribute to be the underlying mechanism regulating a hyperimmune response observed following the pandemic COVID-19 infection.

Until recently, it was not widely known that platelets retain the functional ability to act as mechano-scavengers that collect and bundle bacteria for removal. Research has now solidified a central role for platelets in modulating the first stages of immune response (innate immunity) against bacterial and viral pathogens. Through an elegant series of experiments characterizing the mechanisms by which platelets actively undergo migration in situ, it has been reported that platelets utilize adhesion receptors to mechanically probe the adhesive substrate in their local microenvironment, and through actomyosin-dependent traction forces, platelets were shown to identify and bundle foreign material encountered on the ECM substrata. It has been additionally demonstrated migrating platelet function to collect bacteria microbes, which thereby boosts the activity of professional phagocytes through activation and education of neutrophils and lymphocytes. This mechanism of integrin-mediated locomotion provides new insight into the novel role played by platelets as effector cells and goes against conventional wisdom in which white blood cells are the immune system's first response.

Prior to this past year, it is not widely known for the platelet to initiate immune effector function in the fight against viral invasion. While indirect evidence was in existence in the early '90s demonstrating an association of viral RNA with the platelet fraction post-centrifugation of whole blood, but only recently has there been presented direct evidence of platelet engagement of viral particles. Both platelets and their parent megakaryocytes express immune cell lectin and toll-like receptors to be used as a defense system for deployment against viral pathogen invasion. Utilizing transmission electron microscopy, it has been revealed for the first-time evidence for platelet “phagocytosis” of influenza particles following in vitro incubation. It has been suggested that during attack, platelets undergo morphologic change to engulf active virus utilizing a toll receptor-mediated process possibly preventing cellular entry to minimize viral replication. Subsequent signaling to and engagement of invading white blood cell neutrophils results in platelet activation and degranulation, ultimately resulting in platelet death (termed platelet dropout or thrombocytpenia). Thrombocytopenia associated with viral infection has always perplexed clinical immunologist, but to date no one has proposed a causal link between platelet dropout, viral burden, and immune-compromised patients or their inability to mount an effective and sustained immunologic response to mitigate viral infection.

During platelet-neutrophil signaling, neutrophil extracellular traps are formed (process known as NETosis) to specifically sequester pathogens for leukocyte-targeted killing, whereby a release of neutrophil DNA creates a fine meshwork to bind up pathogen invaders. This step likely precedes phase 2 interferon immune response. Following the interferon response, a likely phase 3 adaptive immune response is initiated whereby pathogen-derived genetic material and protein peptides are presented to initiate antibody production against said invaders are presented to t-cells. Platelets are already dead at this stage and only leave behind growth factors to promote repair of tissue injury—which brings up the important point that during immune compromise there is a death of factors promoting repair of accute lung injury. This process may ultimately lead to sufficient individual immunity attenuating secondary exposure to the specific and related pathogens. However, when sufficient circulating platelet numbers are not present or a patient is immunocompromised, this individual may acquiesce to bacterial and viral pathogen overload, promoting acute respiratory distress syndrome and potential death.

Chronic lung disease is the third leading cause of death worldwide and progressive loss of lung function often promotes disease severity. In young otherwise healthy adults, viral and bacterial pathogens entering the lungs are swiftly identified and tagged for disposal via innate and adaptive immunity responses. However, as mentioned above, high viral burden inevitably leads to thrombocytopenia, breaking the link at the platelet primary defensive front and potentially increasing the risk for lung injury and mortality via cytokine storm.

COVID-19 is a novel single stranded virus that, before December 2019, had never violated the human immune system. Epidemiology studies may soon reveal that COVID-19 patients with impaired lung function exhibit increased mortality versus otherwise healthy individuals, including physically active older adults. In other words, it is further hypothesized that chronic lung disease produces an immunocompromised microenvironment that is hostile to megakaryocyte engraftment and mature platelet production. Data from Wuhan, China suggest males are twice as likely to exhibit severe reaction to COVID-19 infection, with a mean age at mortality of 70 years old, and not surprisingly, the smoking history of Asian males is well documented. Additionally, epidemiology studies indicate the young population in Korea has been hit particularly hard by COVID-19, which would be interesting to determine if these individuals admitted to a history of vaping induced lung injury and/or smoking. Our hypothesis therefore supports the notion that smokers with chronic lung injury can be classified as being immune compromised, and platelet insufficiency carries greater risk for severe lung injury during viral challenge. Support for this hypothesis comes from the pediatric literature where young children exposed to second-hand smoke were found to exhibit severe pulmonary challenges when exposed to respiratory syncytial virus.

In general, a greater majority of the human population would be considered compromised for one reason or another and therefore may present with decreased number of circulating platelets for defense. The ability of the COVID-19 virus to specifically attack lung epithelial cells, in the absence of sufficient quantity of sentinel platelets, would further compromise an organ that produces 50% of the body's front-line immune defense. Therefore, it is critical to systemically upregulate the circulating platelet numbers so the body does not succumb to viral overload.

Deployment of an inexpensive point-of-care system to harvest and deliver supraphysiological concentration of platelets to lungs that have been adversely impacted by viral infection may offer a novel solution that mitigates development of drug resistance, while at the same time minimizes negative side effects associated with anti-malarial therapies (hydrochloroquine and choloroquine with azithromycin) currently under investigation as prophylactic or active antiviral agents.

Covid-19 promises to become the greatest health care concern of the century as it threatens the safety and wellbeing of the global population. Until spread of the virus is contained and a timely solution identified, protecting against future outbreak, global economic decline is an almost certain outcome. The past three months has laid witness to advancement of the COVID-19 pandemic from Asia to Europe and now to North America. As seasonal change ensues and the winter flu season strikes the Southern hemisphere, Australia and other large population will experience similar fate. COVID-19 has created a significant healthcare problem that not only threatens human safety and well-being but also threatens global economic stability. This pandemic has created a great unmet medical need for which no current medical treatment exists to protect human life, particularly in the case of moderate and severe COVID-19 infection where immunocompromised patients exhibit the greatest risk for increased morbidity and mortality.

### SUMMARY OF THE INVENTION

The present disclosure assembles previously unrecognized findings to formulate a novel hypothesis laying the foundation for the development of effective platelet-targeted therapies to augment host defense mechanisms against invading pathogens. Applicants have uncovered a specific treatment protocol and antiviral platelet dose concentration to help immunocompromised patients afflicted with viral pathogen invaders. The importance of this concept will undoubtedly transform future research efforts in the development of novel antimicrobial, antiviral, and antimalarial treatments.

The present invention provides a rapid point-of-care method for treatment of lung pathologies involving acute and chronic inflammation that may lead to increased risk for dysregulated immune function. Specifically, this method may be applied to patients identified as positive for COVID-19 infection. Within a matter of 30 minutes, the patient can undergo treatment that will enhance the number of circulating, functional platelets that can be harvested and concentrated post-centrifugation for intravenous administration. Within minutes of injection, delivered platelets are retained in the lungs to limit viral burden through platelet-mediated phagocytic action exhibited at the lung interface, ensuring viral packaging for subsequent disposal. This method may be effective in reducing both the spread of COVID-19 and additionally in minimizing severity of disease for individuals with known risk for ARDS. Other embodiments include treatment of individuals with COPD, idiopathic pulmonary fibrosis, asthma, and vaping lung injury. The present embodiments may be applied to immunologic indications where systemic bacterial, protozoa, and fungi infections may be treated as an effective means to eradicate foreign microbes without pharmacologic agents, as a novel method to mitigate multi-drug resistance of microorganisms.

More specifically, the method utilizes oxygen multistep therapy to normalize systemic oxygen delivery to a subject's upper and lower extremities by breathing oxygen delivered in a mask at a specified (high) flow rate while the subject is closely monitored during supervised, limited exercise. The patient undergoes a period of oxygen deprivation followed by rapid oxygen normalization. While known to evoke a marked increase in red blood cell release, the described technique surprisingly has demonstrated a robust increase in circulating platelet numbers of approximately 38%. Once the patient cools down, whole blood is drawn and the patient follows procedures disclosed in the TruDose™ protocol below to create platelet rich plasma comprising viable platelets termed platelet-targeted antiviral therapy.

The current invention describes systems and methods related to the field of medical treatments and more specifically regenerative medicine. The present invention is an antiviral treatment protocol that may include the utilization of artificial intelligence to determine an antiviral concentration of platelet rich plasma (2.5×10⁶ platelets/μL) administered intravenously for antiviral clinical benefits. Various embodiments describe a method using an artificial intelligence software and testing system to improve the accuracy and consistency of obtaining the exact blood calculation needed to produce an antiviral dose of PRP. Depending on the immune state of the patient, the protocol can incorporate a multistep oxygen therapy to enhance platelet counts prior to blood draw. In other embodiments a treatment protocol for intravenous administration is also described. In summary, Applicants describe an invention from unexpected findings and discoveries demonstrating antiviral effects that are the result from using the system and method described herein and in testing various components thereof. The hope is this invention can have positive clinical impact to patients suffering from conditions as a result from viral pathogen invasion.

Collectively, the present invention describes the unexpected findings of a treatment protocol and method that can be used for antiviral benefits. Applicants are unaware of any intravenous PRP administration for antiviral effects.

In various embodiments, systems and methods are provided describing a testing method and artificial intelligence software for obtaining a blood calculation leading to a dose specific therapy of antiviral PRP. The current invention utilizes the Applicant's Harbour Cell Software™, described in U.S. Pub. Pat. App. No. 2019/0088372, which is hereby incorporated by reference, to determine a specific blood calculation. The blood calculation can be determined from following exemplary steps:
Test baseline platelet count via finger stick (capillary sampling);
Calculate exact aspiration volume needed for centrifugation to achieve antiviral target dose;
Test PRP post-centrifugation to confirm requested target dose;
Calculate re-aspiration or dilution volumes to adjust the target dose; and
Verify the target dose/mL has been produced prior to treatment.

As a baseline, the medical community has defined a normal circulating platelet count between the range of 150,000 platelets/μL to 400,000 platelets/μL. However, as stated previously, patients who are immunocompromised have altered platelet counts, which in turn can alter the antiviral target dose intended of 2.5×10⁶ platelets/μL.

In other embodiments, a multistep oxygen therapy can be utilized for patients who are immunocompromised. Unexpectedly, Applicant has uncovered that multistep oxygen therapy can upregulated platelet numbers by a mean of 38% within five minutes. PRP treatments are point of care treatments with a long history of dosing inaccuracies. The ability to test a patient's baseline count, determine how the platelet counts are altered, administer multistep oxygenation prior to blood draw, for the sole purpose of achieving an antiviral PRP dose, represents a novel ability to ensure patients have therapeutic treatments at the time of injection. The novelty of this finding relates to the ability to deliver therapies of greater potency since platelet number should directly correlate to reductions in viral burden.

In various embodiments, parts of this method process can be utilized in similar fashions to determine a blood calculation or platelet calculation. Those skilled in the art can potentially develop additional algorithms, calculations, or testing methods to achieve a similar blood calculation number.

In other embodiments, a Minimum concentration dose of 2.5×10⁶ platelets/A has been discovered by the Applicants to be antiviral. Excessively high platelet counts circulating in the bloodstream are known to be a medical concern, whereby counts exceeding greater than 1.0×10⁶ platelets/μL are widely considered to be life threatening. This fact demonstrates why very few have explored intravenous administration of PRP. While Alcaraz et al (Platelet-Rich Plasma in a Patient with Cerebral Palsy, Am J Case Rep. 2015; 16: 469-472 (Year: 2015), which is incorporate herein by reference) is the first to demonstrate intravenous administration is possible, their clinical findings and exploration was noted to be limited due to their inability of increasing the dose strength/μL beyond 1.25×10⁶ platelets/μL with a minimum of 25-30 mL of treatment volume needed for clinical success. In other words, the findings of Alcarez et al demonstrate a total delivery of approximately 31 million platelets achieves clinical benefits. In contrast, the Applicants' findings delivered half the number of platelets and demonstrated antiviral effects. Therefore, clearly another novelty of the current invention is demonstrating IV PRP is dependent on dose strength of platelets/μL versus the total number of platelets administered.

In other embodiments, a minimum concentration dose of 2.5×10⁶ platelets/μL has been determined to provide antiviral benefits. Applicant's software utilizes proprietary metrics and algorithms for calculations and outcome collections. One of the functions is determining a successful dose to outcome relationship from various patient history and outcome reporting measures. For example, the Applicant submitted a novel method of intravenous administration of PRP for neurological conditions, U.S. Prov. Pat. App. Ser. No. 62/757,702, which is hereby incorporated by reference, after 100 patients reported with unexpected clinical benefits. Shortly into the 100-patient study, a neurological dose was determined by the AI software that is not otherwise known in the public domain. In a similar manner, the current invention may utilize the artificial intelligence to determine a beneficial antiviral dose. In one embodiment, a minimum concentration dose of 2.5×10⁶ platelets/μL delivered in 6 mL produces an antiviral dose-Strength outcome. In various embodiments, artificial intelligence does not have to be utilized.

In other embodiments, systems and methods are provided describing a sequential centrifugation process within this intravenous treatment protocol for obtaining a minimum concentration of, for example, 2.5×10⁶ platelets/μL. Platelet rich plasma (PRP) is prepared through a process known as differential centrifugation whereby the process fractionates the cellular constituents based upon their specific gravity. Currently, there are countless ways to produce PRP. The current treatment protocol utilizes a minimum of two-steps to achieve the supraphysiological concentration of, for example, 2.5×10⁶ platelets/μL in 6 mL of treatment volume.

In various embodiments, the first step prior to centrifugation is determining the correct volume of blood for phlebotomy. The second step is processing this entire volume of blood to reduce and remove red blood cells and produce a supernatant for further processing. The final step is processing the supernatant down to a final concentrate treatment volume minimum of 6 mL.

In various embodiments, a multiple step centrifugation and dilution adjustment process (3, 4, 5, etc.) could achieve a minimum concentration of 2.5×10⁶ platelets/μL.

In other embodiments, an intravenous administration of a minimum of 2.5×10⁶ platelets/μL is administered without an activating agent. Activating agents like citrate or calcium bovine thrombin are widely known and used for site specific/local route injections. Activating agents release platelet growth factors and terminate their activity at about 12 hours after injection, which would not be suitable for systemic circulating viral agents. Applicants' technique did not use an activation agent allowing the body's response to determine the ideal release time point.

In other embodiments, the described technique is an intravenous push administration at a rate of approximately 1 mL per second into the patient's vein, such as an arm vein that does not require drip administration or dilution of anticoagulants. It is common practice for intravenous cell therapy administration to be a drip or diluted technique to minimize the potential for clotting and adverse events. For example, drip administration is commonly used with saline or anticoagulants, like Heparin. Considering platelets are the one cellular component in the body known to form clots, this has discouraged intravenous PRP exploration, especially in supraphysiological concentrations described herein. In various embodiments, the rate of administration can be more or less than 1 mL per second and/or into another venous access other than the arm.

Applicant has developed several testing tools, processing tools, and artificial intelligence (AI) learning software that collectively personalize cell therapies to individual patients using Applicant's software. In various embodiments, the treatments described herein may include a rapid point-of-care method for treatment of lung pathologies involving acute and chronic inflammation such as COPD, idiopathic pulmonary fibrosis, asthma, vaping lung injury, and virulent, potentially deadly forms of influenza initiating cytokine storm. Other immunologic indications where systemic bacterial, protozoa, and fungi infections may be treated by the present method as an effective means to eradicate foreign microbes without pharmacologic agents, as a novel method to mitigate multi-drug resistance of microorganisms, are additionally presented.

The above summary of the invention is not intended to represent each embodiment or every aspect of the present invention. Particular embodiments may include one, some, or none of the listed advantages.

### BRIEF DESCRIPTION OF THE DRAWINGS

A more complete understanding of the method and apparatus of the present invention may be obtained by reference to the following Detailed Description when taken in conjunction with the accompanying Drawings wherein:

FIG. 1 is a flowchart of a method according to an embodiment; and

FIG. 2 is a flowchart of a method according to an embodiment.

### DETAILED DESCRIPTION

The present invention is directed towards systems and methods for increasing successful outcomes in cell therapy treatments. This treatment protocol may include the use of Applicant's Harbour Cell Software m or other device to calculate exact aspiration blood volumes in order to concentrate a consistent PRP minimum dose on the order of 2.5×10⁶ platelets/μL for diseases due to viral pathogen invasion. Furthermore, the PRP is administered intravenously to promote circulatory repair.

One novelty of this treatment protocol includes delivering a consistent PRP dosage to all patients. The Harbour Cell Software™ may be used to determine the exact blood aspiration volume in order to achieve consistency of PRP dosing. First, a baseline platelet count was taken from each respective patient. Next, the baseline platelet count, the centrifuge recapture efficiency, injection volume, and target dose of on the order of 2.5×10⁶ platelets/μL were all used to calculate and display exact aspiration volumes for each patient. The aspiration volumes were highly variable for each patient, but a minimum of 2.5×10⁶ platelets/μL was achieved for this embodiment. In other embodiments, the concentration may be higher or lower than 2.5×10⁶ platelets/μL may be utilized depending on the treatment parameters. The 2.5×10⁶ platelets/μL dose was found to be sufficient to provide antiviral benefits.

Another novelty of this treatment protocol was the intravenous administration of the PRP. PRP is widely known and administered via a local injection to an injured site. Local administration has the drawbacks of prematurely releasing platelet growth factors and peptides, whereas the benefit of the IV administration is the systemic release during circulatory repair. During one study performed by Applicant, nine of the eleven patients received IV administration of surpraphysiologic PRP. All nine of these patients reported improvements to neurocognition, language, memory, eyesight, handwriting, focus, systemic pain relief, inflammation reduction, detoxification, and academic grade improvements. The two patients who received localized injections only reported pain relief at their injured sites. These two patients did not report any other improvements like those reported from the patients receiving the IV treatment.

Various embodiments are directed towards a cell therapy treatment protocol for viruses, such as COVID-19 More specifically, a treatment protocol administering, on the order of, at least 2.5×10⁶ platelets/μL intravenously. In various embodiments, the treatment protocol calls for consistency of platelet dose administrations, which may be achieved by following the systems and methods of, for example, applicant's Harbour Cell Software™ to calculate exact blood aspiration volumes to achieve consistent doses.

Currently, point-of-care cell therapy lacks sufficient standardization. In various embodiments, the systems and methods may utilize the Harbour Cell Software™ for making the latest treatment protocols available to doctors, nurses, and other technicians at the point-of-care. When conducting a cell therapy treatment study in a controlled setting, several safety measures may be in place to ensure accuracy that may not be in place in a real-world setting. In both the research and real-world settings, cell therapy treatments generally include a physician aspirating a determined large volume of autologous fluid from a subject, concentrating this fluid via centrifugation to obtain a final small volume of concentrate, and then injecting this small volume concentrate to a target site. In this treatment protocol embodiment, the final small volume of concentrate is delivered intravenously for treatment.

Clinical outcomes are more likely to succeed when a target concentration is achieved prior to centrifugation. Furthermore, the advancement of cell therapy regenerative medicine can occur when methods and devices insuring quality control and dose consistency are understood and administered. In order for one to be sure enough total cells are present to centrifuge, despite all of the other variables, one must aspirate the appropriate volume from the patient to reach a desired target platelets/μL in the final volume of concentrate. Physicians often determine their aspirating volume by the centrifuge kit volume limitations, habitually aspirate the same volumes for each patient, or stop aspirating when they feel they have enough.

In various embodiments, systems and methods utilizing the Harbour Cell Software™ ensure the appropriate amount of target platelets/μL has been achieved in the final small volume of concentrate. In the present embodiment, antiviral improvements were achieved by delivering consistent target platelets/μL intravenously.

An appropriately dosed PRP therapy may offer new hope in addressing virus-related diseases, such as COVID-19. Epitheilial, endotheilial, and PRP-derived growth factors could address intestinal and vascular permeability causations, as well as, revascularization, Phagocytotic PRP peptides and macrophage reprogramming could not only restore gut dysbiosis, but remove free roaming neurotoxic bacteria, pathogens, and viruses. Furthermore, phagocytosis would address cell-invading small-colony variant bacteria capable of cell mimicry and disruption of cell mitochondria. Remyelination of neuronal axons, via brain derived neurotrophic factor, could restore connectivity and axonal protection from free roaming bacteria/pathogens and Viruses. Lastly, PRP/macrophage induced inflammation would signal an orchestrated tissue repair remodeling cascade (tissue genesis) via paracrine signaling and cell proliferation/differentiation choreographed via platelets and growth factors. Various embodiments of treatment methods may include using Applicant's Harbour Cell Software™ to ensure consistent and accurate results, discovery and refine new treatment methods, and/or supraphysiologic dose administration intravenously of PRP.

To date, nothing within the clinical literature can be found investigating, demonstrating, or reporting the clinical findings described herein and, ultimately, the discovery of a treatment method for intravenous administration of PRP for viral diseases. Furthermore, there are no reports characterizing consistent platelet dosing of treatments for vital diseases.

In various embodiments, the treatment method may include a testing protocol, AI dose learning, and/or an intravenous administration protocol of the PRP. In a first step, various patient demographics may be inputted into the software for learning. Demographics can include, but not limited to, disease state information, health information, objective patient characteristics and lifestyle information. For example, the medications a patient is taken, the patient's history of viral infections, and/or consumption of alcohol, all of which may affect platelet counts, may be inputted. Demographic information may also include genomic and genetic specifics of individual patients. This information is compared against the learning database to determine the degree of impact each demographic variable potentially has on outcome success. Over time, the learning metrics understand the degree of each independent variable impacting outcome success which is then correlated back to dosage.

In a second step, a patients baseline platelet count is established, such as via finger stick (capillary sampling). Following the inputting of demographic information, a capillary sampling of blood may be executed. The blood sample could be sampled and counted different ways known to those skilled in the art. In some embodiments, blood sampling testing kits may be used that contain specific capillary tubes and testing slides to increase accuracy.

In a third step, the exact aspiration volume for centrifugation is calculated. Once the demographic information and baseline platelet count have been inputted into the software, a blood aspiration calculation may be determined and displayed. The learning software may accounts for demographic variability, baseline cell counts, and/or previous recorded outcomes to determine an overall dose-to-outcome relationship. For example, the software may have learned that a minimum dose/mL is needed for success of a treatment for a certain disease. For a patient presenting with that disease and having several demographic variables that impact baseline cell counts, the software may be able to adjust accordingly. Baseline cell count is one of several variables that impact dosing inaccuracy. Additionally, other variable can impact the calculations. For example, individual operators (human error) have different variabilities of performance that are often unavoidable. The artificial intelligence software may learn individual operator performance deviations and may include that in the various metrics factored into patient aspiration calculations. Based on these collective learning metrics and algorithms, the software may then calculate the patient-specific blood aspiration volume needed to produce the minimum consistent therapeutic dose/mL for the disease treatment.

At a fourth step, target dose is tested post-centrifugation for re-aspiration or dilution adjustments. Following centrifugation, the testing protocol may call for confirmation of the target dose. A sample of the final concentrate is collected and a measurement is initiated. Once the final measurement is inputted into the software, dilution calculations or re-aspiration calculations are performed. By way of example, in real-world patient encounters, Applicant's software has achieved better than 95% accuracy in calculating a blood aspiration that will produce a desired target dose.

At a fifth step, re-aspiration for centrifugation or dilution adjustments to reach desired target dose are performed. It is often easier to dilute than to re-aspirate, re-centrifuge, homogenize to final concentrates, re-test and verify. Therefore, the learning metrics can be defaulted to err on the side of over aspiration to avoid those time-consuming processes. At a sixth step, the treatment therapy is administered. At a seventh step, patient follow up and documentation of validated outcomes are measured. The determination of a dose-to-outcome response is dependent on the outcome success of the treatment. Outcome success is collected by the scientific and medical community via validated measurements scales depending on the disease and condition. For example, the pain VAS measurement scale is a unidimensional measure of pain intensity, which has been widely used in diverse adult populations for various treatments and outcome measurements. The change in a patient's pain measurement is an example of a successful treatment outcome metric that may be incorporated into the learning algorithms intended to learn a dose-to-outcome response.

Using the learning software, Applicant has discovered new dosages for treating viral diseases. In one embodiment, the preparation of the PRP may involve a blood phlebotomy and centrifugation. The blood phlebotomy may be executed using World Health Organization guidelines via a butterfly needle, tourniquet, stopcocks, and multiple syringes. The centrifugation preparation process may involve a two-step centrifugation process for PRP sedimentation. In order to produce a supraphysiological concentration of platelets per μL may require a two-step process. Many clinicians only execute a one-step centrifugation protocol to produce PRP, which is often not capable of producing high concentrations of platelets. There are many two-step PRP processing kits commercially available. For example, the Pure PRP system from Emcyte Corporation is a commercially available PRP kit that involves a two-step preparation process. The Pure PRP system first uses a canister for RBC and platelet supernatant sedimentation. Following the first centrifugation spin, the operator withdraws the lighter supernatant and injects this into the second and final processing canister for separation. The second centrifugation spin separates the platelet supernatant into platelet poor plasma and platelet rich plasma. Once centrifugation is finished, the operator removes a volume of platelet poor plasma and continues by homogenizing the remaining volume. The volume of platelet poor plasma can be a various percentage depending on the final volume of PRP needed for injection. The percentage can be anywhere from 1-95% of the total volume being removed. Depending on the calculated blood amount, either one or two cylindrical canisters may need to be filled with an appropriate amount of blood. If only one canister was used, a counterweight may be used to balance the first canister. In either scenario, the first centrifugation spin may be executed for two minutes at 3800 rpm to isolate red blood cells and platelet supernatant. The platelet supernatant may then be removed and placed in a third canister for isolation of platelet poor plasma and platelet rich plasma. Whether a single or double canister, the second spin may be executed for seven minutes at 3800 rpm to produce a final PRP concentrate of a minimum 2.0×10⁶ platelets/μL. Following centrifugation, a small aliquot of PRP may be removed to confirm a 2.5×10⁶ platelets/μL dose. In trial cases, the minimum 2.5×10⁶ platelets/μL was achieved and an injection volume of 6 mL was infused to patients intravenously at 1 mL/second. In other cases, the concentration was increased to 3.0×10⁶ platelets/μL and in other cases, to, on the order of, 3.5×10⁶ platelets/μL or higher. In some embodiments, the dose was administered at 0.5 mL/second or lower, while in others it was administered at 1.5 mL/second, 2.0 mL/second or higher. In some embodiments, the injection volume was between about 5 mL to 7 mL, while in others it was reduced to below 5 mL, while in others it was increased to above 7 mL.

In some variations, the PRP preparation protocol could be a variation of centrifugation times and forces for both the RBC sedimentation and PRP sedimentation. Times can range anywhere from 1-20 minutes or more depending on the step of the process. Forces can range anywhere from using gravity to a centrifugation force up to 100,000 rpm. The time needed for sedimentation in the two steps would be proportional to the forces being used.

In some variations, the PRP processing components can be a variety of shapes and sizes. PRP processing components can also be a commercially available PRP kit or a combination of assembled components. For example, a minimum 2.5×10⁶ platelets/μL dose may be achieved with laboratory conical tubes. The conical tubes may require a larger calculated amount of blood draw, however, a two-step centrifugation process may still apply, as well as, determined time and centrifugation force. In either scenario, the performance of the commercial processing kit or manual processing method is one of the variables of blood aspiration calculations. The learning software algorithms learn the standard deviation of performance for the method being used to help calculate consistent PRP doses.

In some variations, the PRP preparation process involves a human operator whereby introducing human error. Any additional errors affect blood aspiration calculations which ultimately impact consistency of the dose. For example; a first operator could add an additional 10% of error compared to a second, more skilled, operator. The learning software metrics learn the proficiency of different operators as another variable influencing the consistency of dosing.

In another embodiment, a small aliquot sample is used for measurement to determine the minimum 2.5×10⁶ platelets/μL has been achieved. Following centrifugation and homogenization of final concentrate sample, a transfer cup may be used to procure, for example, a 5 μL sample for analysis and measurement. The volume of measurement could be various volumes depending on the measurement instrument being used.

In another embodiment, a small aliquot sample is used for measurement to determine the patient's baseline platelet count. The baseline platelet count is another variable factored into the intelligent aspiration calculations. For example, a 20 μL capillary sample may be taken and used for measurement. The volume of measurement could be various volumes depending on the measurement instrument being used.

In some variations, the PRP compositions of each individual may comprise of varying concentrations of various types of white blood cells, lymphocytes, monocytes, eosinophils above their respective baseline counts. These concentrations over baseline are typically reported as “times baseline.” For example, the concentrations may vary between 1×-10× over their respective baseline. The concentrations of lymphocytes and monocytes may be between about 1.1 and about 2 times baseline, about 2 and about 4 times baseline, about 4 and about 6 times baseline, about 6 and about 8 times baseline, or higher. The concentrations of eosinophils in the PRP composition may be about 1.5 times baseline. In some variations, the lymphocyte concentration is between about 5,000 and about 20,000 per μL and the monocyte concentration is between about 1,000 and about 5,000 per μL. The eosinophil may be between about 200 and about 1,000 per μL.

In certain variations, the PRP composition may contain a specific concentration of neutrophils. The concentration may vary between less than the baseline concentration of neutrophils to eight times the baseline concentration of neutrophils. In some variations, the neutrophil concentration may be between 0 and about 0.1 times baseline, about 0.1 and about 0.5 times baseline, about 0.5 and 1.0 times baseline, about 1.0 and about 2 times baseline, about 2 and about 4 times baseline, about 4 and about 6 times baseline, about 6 and about 8 times baseline, or higher. The neutrophil concentration may additionally or alternatively be specified relative to the concentration of the lymphocytes and/or the monocytes. In preferred embodiments, the neutrophil concentration is less than the concentration in whole blood. In other embodiments, the neutrophil concentration is 0.1 to 0.9 the concentration found in whole blood, yet more preferably less than 0.1 the concentration found in Whole blood. In other embodiments, the neutrophils are eliminated or non-detectable in the PRP composition.

The results from this study proves the novelty of the intelligence software and ultimately the novelty of the treatment methods described herein. Dosing inaccuracy and lack of standardization is a widely known problem within the scientific literature, especially PRP. Because there has been a lack of tools to help prepare consistent dosing, treatment protocol discovery has stalled in the regenerative medicine field. Reducing Applicant's Harbour Cell Software to practice led to unexpected clinical findings and the discovery of novel antiviral treatment protocols, including supraphysiologic dose administration intravenously of PRP.

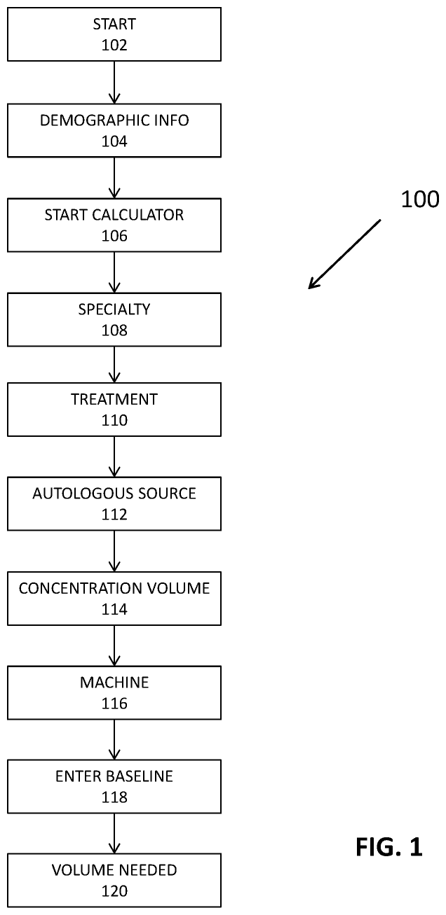

Referring now to FIG. 1, a flowchart is provided of an embodiment of a method 100 of providing a treatment protocol using a user device. At step 102, a user is prompted to select whether the use will be for research purposes or for non-research purposes. To various embodiments, at step 104, patient information and/or de-identified demographics of the intended subject could be inputted or selected from a drop down menu, such as, for example, whether the subject is human or animal, the sex of the subject, age, name, initials, or other indicia, treating physician, facility, location, and other relevant information. Such information may be useful for tracking, data and research collection purposes. These selections may be inputted and displayed via the user device.

At step 106, the user begins the calculation workflow. At step 108, the user device receives input from the physician regarding specialty. If being used for non-research purposes, then the user device may be programmed to prompt specialty selections that are within the published literature showing cell therapy human outcomes within the specified specialty. If the specialty is not reported in published literature or not published with human outcomes, then, at step 108, the physician may be prompted to add the specialty before proceeding through the workflow prompt. When a new specialty is added, the physician may be notified by the user device that it will no longer be accessing the cloud based and/or embedded published outcomes. The device may still proceed through the workflow prompts and calculate the needed autologous volume, however, the physician may be prompted that the volume calculated is intended for an experimental specialty use not reported in the published literature.

In the current treatment protocol embodiments, the physician specially would fall under those pertaining or who treat viral infections and/or lung disorders. The Harbour Cell Software™ may be utilized for research purposes, and thus falling into the category of not reported in published literature.

At step 110, the device receives input from the physician regarding an intended treatment. If being used for non-research purposes, then the device may prompt treatment selections based at least in part on the specialty selection that are within published literature showing cell therapy human outcomes. If the intended treatment is not reported in published literature or not published with human outcomes, then the physician will be prompted to add the treatment before proceeding through the workflow prompt. If a treatment is added, then the physician may be notified by the machine that it will no longer be accessing the cloud based and/or embedded published outcomes. The device may, still proceed through the workflow prompts and calculate the needed autologous volume, however, the physician may be prompted that the volume calculated is intended for an experimental treatment use not reported in the published literature. The physician may need to acknowledge this before proceeding to the next workflow prompt. If the device is being used for research purposes, then the physician may input the designated specialty.

In the current treatment protocol embodiment, IV dose administered PRP treatment is not reported in published literature and therefore must be inputted into the software before proceeding with the workflow prompts.

At step 112, the device receives input from the physician regarding an intended autologous source and strength number. The Harbour Cell Software™ incorporates these attributes as part of the workflow so physicians can be informed of the most current published methods during the course of treatment workflow. In the current embodiment, this treatment protocol discovery falls into the experimental treatment workflow prompts that are not reported in literature. The autologous source entered is autologous blood-PRP.

At step 114, the device receives input from the physician regarding, concentration volume needed. If being used for non-research purposes, the device may only prompt a default numerical concentration milliliter volume, determined from the treatment selection. The defaulted volume may be based at least in part on the relevant published literature. The treatment targeted cell range per μL is displayed for the physician to view and confirm. In the current treatment protocol embodiment, a minimum of, on the order of, 2.5×10⁶ platelets/μL was inputted as the target as there is no established target range of virus treatment. The physician may have the option to increase or decrease the concentration volume by selecting plus (+) and minus (−) signs. The value for the starting volume needed to achieve the concentration volume needed for treatment is calculated based at least in part on the different inputs received during the workflow. Any number of calculations can be used to determine starting volume needed to maintain target platelets/μL necessary for the intended treatment. Changing various inputted values will affect the resulting calculations.

At step 116, the device receives input from the physician regarding the concentration machine being used. The performance criteria is another example of a value that may influence the starting volume calculation. This is due to the known studied performance variabilities of commercial cell concentration devices. The physician may have options to add a machine. When adding a machine, a weighted performance average may be calculated to determine the starting volume calculation. In this scenario, the device may notify the physician that a weighted average is being used to determine the final calculations and not a known performance for the added machine. In the current treatment protocol embodiment, an established commercial centrifuge was utilized with similar performance characteristics and weighted average calculations to other known commercial centrifuges. These machine performance criteria were utilized to determine the final aspiration calculations to achieve consistent PRP concentrate yields of 2.5×10⁶ platelets/μL for this particular concentration device.

At step 118, the user device receives baseline cell numbers that will be used for calculations. The baseline cell number can be taken by any commercially available cell counter, platelet counter, hemocytometer, or like device. The user device may receive the baseline numbers via manual input or via wired or wireless connection to the counter and/or other backend system to determine which calculation should be performed: for platelets, RBCs, HSCs, WBCs, exosomes, adipose pre-cursor cells, or MSCs. The user device can also be connected, either wired or wirelessly, to capable cell counting devices in order to transfer baseline data instead of manual input. These selections are displayed and received via the user device. Information provided by the system based at least in part on user inputs may assist the user in determining the source material to be used in the treatment. Because the baseline may vary depending on the source material, in various embodiments, although not required, it may be preferable for a user to input other information (e.g., specialty, treatment, and/or autologous source) before determining and/or inputting the baseline cell number. In the current treatment embodiment shown, the baseline platelet number is inputted after various other information has been entered.

At step 120, the device displays the starting volume amount of autologous source volume needed to be aspirated from the subject. This final calculation is determined based at least in part on the previous inputs from the clinician. This starting volume is the final calculated volume needed from the individual subject, to be concentrated, in order to concentrate a final treatment volume containing a minimum of 2.5×10⁶ platelets/μL range.

In some embodiments, the device may also determine dilution and/or hyperconcentration calculations. Dilution and/or hyperconcentraton calculations may be an important value to know following machine concentration. By way of example, in various embodiments of the system, the starting volume was calculated to ensure an appropriate target platelets/μL treatment yield is achieved. Following machine concentration, a physician can test a small aliquot sample of the concentrate. If the platelets/μL volume exceeds the intended target, the excess plasma or other fraction of the separation can be added to dilute the concentrate in order to achieve the intended target platelets/μL. The treatment system may calculate exactly how much dilution should be added. The opposite would occur if too little target platelets/μL were achieved. If the target platelets/μL is less than the intended target treatment need, then hyperconcentration would need to occur. In this case, a cell analysis of the concentrate would be used by the system to make the hyperconcentration calculations_. The system would calculate the amount of excess plasma or separated fraction to be removed from the concentrate. This would yield a total volume less than the desired treatment volume, but would be at the desired treatment target platelets/μL. Any number of weighted algorithms and calculations could be used to determine the final volume needed or other values within the equation. In the current treatment protocol embodiment, a minimum of 2.5×10⁶ platelets/μL was achieved before administering.

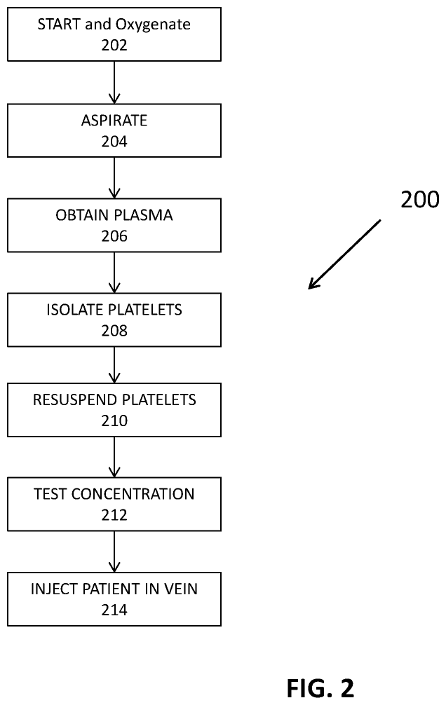

Referring now to FIG. 2, a PRP treatment method 200 is provided. At step 202 the method begins. In one embodiment of the treatment protocol, whole blood is aspirated from a patient at step 204. Next, at step 206, the whole blood is concentrated by obtaining a plasma fraction of the whole blood and isolating the platelets therefrom at step 208. Then the platelets are re-suspended at step 210 and the concentration of the final concentrate is tested at step 212. Finally, the PRP concentrate is administered via intravenous injection under time-controlled administration (approximately 1 mL per second) at step 214. Prior to IV injection, careful filtering of any impurities was undertaken to the PRP concentrate. Dose specific PRP-IV administration is indeed novel due to PRP being historically delivered only at the injury site. The novel aspect of the PRP-IV administration is the theoretical homing phenomenon whereby cells migrate to the organ of their origin or to repair. With viral and lung diseases, there are many underlying repair needs. The body releases platelets during times of repair. The current IV circulatory repair approach allowed high concentrations of platelets to home to various sites of repair before orchestrating a paracrine repair process and release of platelet growth factors. The current treatment protocol approach proved this as evident by patients reporting more than one improvement in lung functions. In contrast to historic PRP site injections, growth factors are released immediately and focused to a specific injury. Again, this proved evident as the two patients who received site injections did not report any other benefits than the injury site pain relief. Therefore, PRP-IV administration promoting systemic circulatory repair proved beneficial.

In another aspect, the proposed treatment protocol adhered to a specific minimum dose concentration for each patient. Currently, there are no devices for PRP cell therapy that provide a method of quality control and standardization like the Harbour Cell Software™. In the absence of such device and standardization, physicians are either delivering suboptimal treatments or potentially inhibitory treatments. The lack of standardization and quality control inhibits the advancement of cell therapy knowledge. For example, a concentration of 3 billion platelets per MI: may be inhibitory to tenocyte and neo-vascularization behavior. Knowing the optimal concentration range is not helpful unless the amount of injected PRP volume, the concentration machine used, the starting volumes used to concentrate, and/or the absolute value of platelets is also known. Aspirating the same amount of blood in each patient is not a scientifically sound way to reach the optimal concentration range. For example, if one patient has a baseline of 150,000 platelets/μL and another has a baseline of 350,000 platelets/μL, then aspirating the same volume creates a significant variable. The current treatment protocol embodiment uses the Harbour Cell Software™ to eliminate the inherent variabilities, deliver consistent doses, and prohibit inhibitory concentrations.

Although various embodiments of the method and apparatus of the present invention have been illustrated in the accompanying Drawings and described in the foregoing Detailed Description, it will be understood that the invention is not limited to the embodiments disclosed, but is capable of numerous rearrangements, modifications, and substitutions without departing from the spirit and scope of the invention.

What is claimed is:

1. A method of treating a disorder comprising:
identifying a disease disorder in a patient with underlying causation of viral invasion;
intravenously injecting about 6 mL of a platelet-rich plasma composition having a concentration of at least about 2.5×10⁶ platelets/μL into the patient to produce antiviral improvements in the patient;
wherein the platelet-rich plasma composition is injected into a vein of the patient at a rate of about 1 mL/second; and
wherein the platelet-rich plasma composition does not include an activating agent.

2. The method of claim 1, wherein a process for oxygenating the patient's blood is undertaken prior to aspirating the patient's blood to create the platelet-rich plasma composition.

3. The method of claim 1, wherein the platelet-rich plasma composition does not include a dilutant.

4. The method of claim 1, wherein the platelet-rich plasma composition does not include a saline dilutant.

5. The method of claim 1, wherein the platelet-rich plasma composition does not include an anticoagulant.

6. The method of claim 1, further comprising preparing the platelet-rich plasma composition from whole blood of the patient.

7. The method of claim 6, wherein preparing the platelet-rich plasma composition from the whole blood comprises the steps of:
obtaining a plasma fraction from the whole blood;
isolating platelets from the plasma fraction;
resuspending the platelets in a reduced amount of plasma; and
wherein an activator of the platelets is not added to the platelet-rich plasma composition.

8. The method of claim 1, further comprising testing the concentration of the platelet-rich plasma composition prior to injection.

9. A medical treatment protocol for treating viral pathogen invasion in a human comprising:
determining a baseline platelet concentration of a blood sample of a patient;
determining a volume of blood to aspirate from the patient for a platelet-rich plasma treatment based at least in part on (a) a concentration target of 2.5×10⁶ platelets/μL, (b) the baseline platelet concentration, and (c) a treatment volume target of 6 mL of final concentrate;
aspirating the volume of blood from the patient;
concentrating the aspirated blood to obtain a treatment volume of at least about 6 mL of the final concentrate having a concentration of at least about 2.5×10⁶ platelets/μL; and
injecting the final concentrate into the patient intravenously at a rate of about 1 mL/second thereby alleviating at least one symptom of a neurological disorder.

10. The protocol of claim 9, wherein the concentrating involves a two-step centrifugation process.

11. The protocol of claim 9, wherein the final concentrate does not include a dilutant.

12. The protocol of claim 9, wherein the final concentrate does not include a saline dilutant.

13. The protocol of claim 9, wherein the final concentrate does not include an anticoagulant.

14. The protocol of claim 9, wherein concentrating the aspirated blood comprises the steps of:
obtaining a plasma fraction from the aspirated blood;
isolating platelets from the plasma fraction;
resuspending the platelets in a reduced amount of plasma; and
wherein an activator of the platelets is not added to the final concentrate.

15. The protocol of claim 9, further comprising testing the concentration of the final concentrate prior to injection.

16. A method for administering a medical treatment comprising:
determining a baseline platelet concentration of a blood sample of a patient;
receiving an indication of a treatment volume of concentrate to be used in a cell-therapy treatment;
calculating an aspiration volume of blood to be aspirated for the cell-therapy treatment to achieve a platelet concentration target range of 2.5×10⁶ platelets/μL, based at least in part on the baseline platelet concentration for the patient and the indicated treatment volume of platelet-rich plasma concentrate;
aspirating the volume of blood from the patient;
concentrating the aspirated blood to obtain a treatment volume of at least about 6 mL of the platelet-rich plasma concentrate having a concentration of at least about 2.5×10⁶ platelets/μL; and
injecting the platelet-rich plasma concentrate into the patient intravenously at a rate of about 1 mL/second.

17. The method of claim 16, wherein the concentration of the platelet-rich plasma concentrate is about 3×10⁶ platelets/μL.

18. The method of claim 16, wherein concentrating the aspirated blood comprises the steps of:
obtaining a plasma fraction from the aspirated blood;
isolating platelets from the plasma fraction;
resuspending the platelets in a reduced amount of plasma; and
wherein an activator of the platelets is not added to the platelet-rich plasma concentrate.

19. The method of claim 16, wherein the platelet-rich plasma concentrate does not include a saline dilutant.

20. The method of claim 16, wherein the platelet-rich plasma includes viable functional platelets.

21. The method of claim 16, wherein the platelet-rich plasma concentrate is used to treat viral infections and other acute and chronic immunologic disorders, including septic bacterial infection.

22. The method of claim 16, wherein the platelet-rich plasma concentrate reduces viral burden without harmful side effects of pharmacologic agents to mitigate evolution of drug resistant microorganisms.

23. The method of claim 16, wherein the platelet-rich plasma concentrate is used to treat one or more of: HIV-AIDS, SARS, MERS, H1N1, covid-19 and related viruses.

24. The method of claim 16, wherein the platelet-rich plasma concentrate is used to promote lung repair in individuals with e-cigarette or vaping associated lung injury.

25. The method of claim 16, wherein the platelet-rich plasma concentrate is used to promotes lung repair in individuals with COPD, idiopathic pulmonary fibrosis.

26. The method of claim 16, wherein the platelet-rich plasma concentrate is used to provide a rapid point-of-care method for treatment of lung pathologies involving acute and chronic inflammation such as COPD, idiopathic pulmonary fibrosis, asthma, vaping lung injury, and virulent, potentially deadly forms of influenza initiating cytokine storm.

27. The method of claim 16, wherein the platelet-rich plasma concentrate is used to treat systemic bacterial, protozoa, and fungi infections by eradicating foreign microbes without pharmacologic agents to mitigate multi-drug resistance of microorganisms.
